(19) United States
(12) Patent Application Publication
(10) Pub. No.: US 2016/0045449 A1
(43) Pub. Date: Feb. 18, 2016

(54) Formulations and Methods for Attenuating Respiratory Depression Induced by Opioid Overdose

(71) Applicant: Alpharma Pharmaceuticals LLC, Bridgewater, NJ (US)

(72) Inventors: Michael J. Lamson, Cary, NC (US); Veeraindar Goli, Raleigh, NC (US)

(21) Appl. No.: 14/922,474

(22) Filed: Oct. 26, 2015

Related U.S. Application Data
(63) Continuation of application No. 13/881,758, filed on Oct. 17, 2013, now abandoned.
(60) Provisional application No. 61/406,752, filed on Oct. 26, 2010.

(51) Int. Cl. A61K 9/50; A61K 31/485

(52) U.S. Cl. CPC A61K 9/5078; A61K 31/485

(57) ABSTRACT

The invention relates to compositions and methods for attenuating opioid induced respiratory depression. Such compositions comprise opioids and sequestered opioid antagonists in a multi-particulate dosage formulation.

### BACKGROUND OF THE INVENTION

King Pharmaceuticals' deactacore platform, the incorporation of sequestered naltrexone into the core of a controlled-release opioid dosage form which is released only upon disruption of the sequestering polymer matrix, was developed as a means of reducing the effect of excess opioid and drug liking when the product is misused or abused. The deactacore technology is described in detail in U.S. Pat. Nos. 7,682,633 and 7,682,634 US patent Publication Nos. US 20080233156, US 20090131466, US 20040131552, US 20100152221, US 20100151014 and US 20100143483 and PCT Application Nos. PCT/US08/087030 PCT/US08/087043, PCT/US08/87047, and PCT/US08/087055 incorporated herein by reference.

The analgesic drug Embeda® (also referred to as ALO-01) is an example a marketed drug formulation incorporating the deactacore technology. (Prescribing Information: Embeda® (morphine sulfate and naltrexone hydrochloride) extended-release capsules. Alpharma Pharmaceuticals LLC, a wholly owned subsidiary of King Pharmaceuticals, Inc., Bristol, Tenn. June 2009). Commercialized in 2009, Embeda® is a capsule formulation containing controlled-release pellets that release therapeutic amounts of morphine sulfate slowly over time. Naltrexone HCl is sequestered in the inner core in a 1:20 ratio with morphine and released only when the sequestering polymer matrix is disrupted. When taken whole, the inner core remains intact and naltrexone does not affect the analgesic potential of morphine. However, when Embeda® is chewed, crushed, or otherwise physically manipulated, naltrexone is released, absorbed orally, and binds competitively to the mu-opioid receptor, thereby abating or diminishing the euphoric effects of the morphine.

The amount of naltrexone in the deactacore platform varies depending on the potency of the opioid analgesic. Embeda utilizes 4% naltrexone (morphine and naltrexone in a 20:1 ratio). Studies have demonstrated that 12% naltrexone or more may be optimal for oxycodone and hydrocodone. While dose response with respect to euphoria and drug liking in combinations of opioids and opioid antagonists has been explored, little is known about the naltrexone dose response relationship with respect to other pharmacological effects of opioids, including the primary mechanism of fatal opioid overdose: respiratory depression. (White J M and Irvine R J. Mechanisms of fatal opioid overdose. Addiction. 1999; 94(7):961-72; Dahan A, Aarts L, and Smith T W. Incidence, reversal, and prevention of opioid-induced respiratory depression. Anesthesiology. 2010; 112:226-38).

Currently, naloxone is the drug of choice for therapeutic use as a rescue medication in the rapid reversal of opioid-induced activity and adverse reactions. (Longnecker D E, Grazis P A, and Eggers G W N. Naloxone for antagonism of morphine-induced respiratory depression. Anesthesia and Analgesia Current Researches 1973; 52(3):447-53). Administered parenterally, naloxone's pharmacodynamic effects with respect to reversing opioid-induced respiratory depression have been well characterized. (Yassen A, Olofsen E, van Dorp E, Sarton E, Teppema L, Danhof M, and Dahan A. Mechanism-based pharmacokinetic-pharmacodynamic modelling of the reversal of buprenorphine-induced respiratory depression by naloxone. Clin Pharmacokinet. 2007; 46(11):965-80; Kaufman R D, Gabthuler M L, and Beilville W. Potency, duration of action and pA₂ in man of intravenous naloxone measured by reversal of morphine-depressed respiration. J of Pharmacol and Exp Ther. 1981; 219:156-62 In known or suspected opioid overdosage, the usual IV dose of naloxone is 0.4-2 mg to reverse opioid-induced respiratory depression. (Amercian Hospital Formulary Services (AHFS) Information. Naloxone hydrochloride. 2003:2088-89). This initial infusion can be supplemented by multiple injections of noloxone at frequent intervals or with a continuous intravenous infusion. In a post-operative setting, a bolus dose of naloxone can be supplemented with a continuous IV infusion of naloxone 3.7 mcg/kg per hour to reverse respiratory depression.

U.S. Pat. No. 5,834,477 describes compositions of a homogeneous mixture containing both opioid agonist and antagonist which induce minimal respiratory depression. The patent describes the use of sufentanil oxalate and nalmefene in a molar ratio of 15:1.

The effects of a combination of hydrocodone bitartrate and naltrexone hydrochloride on respiratory depression in rats have been assessed. (K. Hew, S. Mason, and H. Penton, A Respiratory Safety Pharmacology Assessment of Hydrocodone Bitartrate and Naltrexone Hydrochloride). A comparison of oxycodone and morphine with respect to respiratory depression in patients has been conducted (Change et. al., A comparison of the respiratory effects of oxycodone versus morphine: a randomized, double-blind, placebo controlled investigation, Anaesthesia 2010.) This study determined that of the extent and speed of onset of oxycodone induced respiratory depression was dose dependent and greater than an equivalent dose of morphine.

Using naltrexone as a rescue medication in humans is a novel use for this drug, as naltrexone is primarily administered orally and chronically to treat opiate and alcohol dependence. When not sequestered in the deactacore formulation, for example after crushing or chewing the formulation and then ingesting, the naltrexone is absorbed at least as rapidly as the opioid (FIG. 2), although opioid persists longer than naltrexone. This would suggest that naltrexone has as much of a potential to prevent respiratory depression in an acute opioid overdose situation as it would in either reversing it or abating it, depending upon the amount of each drug absorbed. Therefore, developing a better understanding of the dose-response relationship between naltrexone and opioid-induced respiratory depression is a question of clinical importance.

### SUMMARY OF THE INVENTION

The present invention relates to opioid compositions comprising a sequestered opioid antagonist that when ingested after tampering (e.g. crushing, chewing or dissolving), release the opioid antagonist and attenuate respiratory depression when administered or ingested after tampering. The compositions of the present invention comprise opiate analgesic drug formulations comprising a solid, controlled release, oral dosage form comprising a plurality of multi-layer pellets, each pellet comprising a water soluble core, an antagonist layer comprising naltrexone or a pharmaceutically acceptable salt of naltrexone coating the core, a sequestering polymer layer coating the antagonist layer, an agonist layer comprising opioid or a pharmaceutically acceptable salt of the opioid coating the sequestering polymer layer, and a controlled release layer coating the agonist layer. When the compositions are administered to a human intact, which means that the compositions have not been tampered with, substantially all of the naltrexone remains sequestered. If however the compositions are tampered with, which means the composition has been crushed, chewed, dissolved, or otherwise altered so that the naltrexone and opioid in the composition have been released from the original dosage form, the compositions have sufficient naltrexone to attenuate opioid-mediated respiratory depression in an individual that has taken the tampered form of the compositions.

The present invention relates to opiate analgesic drug formulations comprising a solid, controlled release, oral dosage form comprising a plurality of multi-layer pellets, each pellet comprising a water soluble core, an antagonist layer comprising naltrexone or a pharmaceutically acceptable salt of naltrexone coating the core, a sequestering polymer layer coating the antagonist layer, an agonist layer comprising an opioid or a pharmaceutically acceptable salt of an opioid coating the sequestering polymer layer, and a controlled release layer coating the agonist layer where substantially no naltrexone or a pharmaceutically acceptable salt of naltrexone is released when administered intact to a human and wherein minimal respiratory depression is induced in a human when the formulation has been tampered with prior to administration to the human.

The present invention also relates to methods of attenuating drug-mediated respiratory depression in a human, incident to the administration to the human of a respiratory depression-mediating drug, wherein the method comprises administering to the human an opiate analgesic drug formulation comprising a solid, controlled release, oral dosage form comprising a plurality of multi-layer pellets, each pellet comprising a water soluble core, an antagonist layer comprising naltrexone or a pharmaceutically acceptable salt of naltrexone coating the core, a sequestering polymer layer coating the antagonist layer, an agonist layer comprising an opioid or a pharmaceutically acceptable salt of an opioid coating the sequestering polymer layer, and a controlled release layer coating the agonist layer.

### FIGURES

FIG. 1. Graph comparing the plasma concentrations of naloxone and naltrexone following IV therapy with naloxone (red) and upon complete release from an 80 mg oral dose of ALO-02 or ALO-04 containing 12% naltrexone (blue).

FIG. 2. Graph comparing the plasma concentrations of naltrexone and oxycodone following a theoretical crushed dose of ALO-02 containing 80 mg of oxycodone and 12% (9.6 mg) of naltrexone.

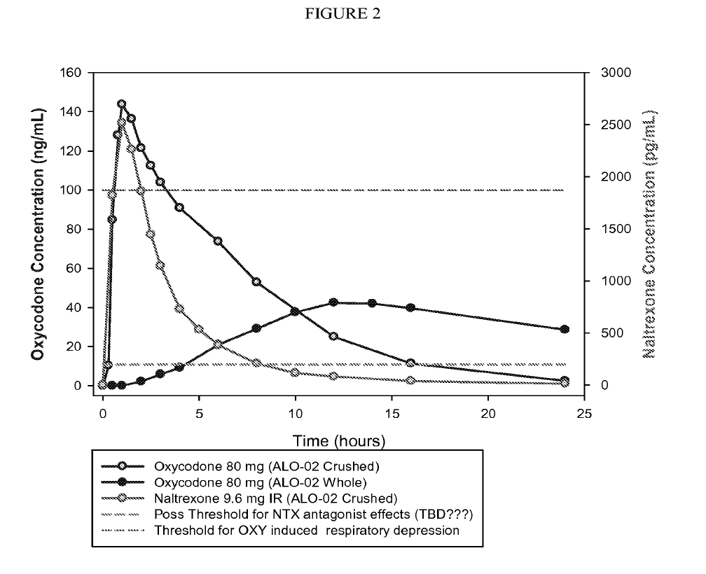

FIG. 3. Graph of modified rebreathing ventilatory response

FIG. 4. Graph of mean (±SD) Eₘₐₓ Values for End Tidal CO₂ by Treatment

FIG. 5. Graph of mean (+/−SE) oxygen saturation (SpO₂) levels over time determined from pulse oximetry following oral administration of oxycodone 60 mg, oxycodone 60 mg+naltrexone 7.2 mg (12%—the current ratio of naltrexone in ALO-02), and placebo.

### DETAILED DESCRIPTION OF THE INVENTION

Provided herein are compositions and methods for administering a composition comprising multiple active agents to a mammal in a form and manner that minimizes the effects of either active agent upon the other in vivo. In particular, the present invention relates to opioid compositions that attenuate respiratory depression when administered to a human. In certain embodiments, at least two active agents are formulated as part of a pharmaceutical composition. A first active opioid agent may provide a therapeutic effect in vivo. The second active agent may be an antagonist of the first active agent, and may be useful in attenuating respiratory depression if the composition is tampered with. The composition remains intact during normal usage by patients and the antagonist is not released. However, upon tampering with the composition (e.g. crushing, chewing, or dissolving the composition), the antagonist may be released thereby preventing, abating or attenuating the opioid from inducing significant respiratory depression. In certain embodiments, the active agents are both contained within a single unit, such as a pellet or bead, in the form of layers. The active agents may be formulated with a substantially impermeable barrier as, for example, a controlled-release composition, such that release of the antagonist from the composition is minimized. In certain embodiments, the antagonist is released in in vitro assays but is substantially not released in vivo. In vitro and in vivo release of the active agent from the composition may be measured by any of several well-known techniques. For instance, in vivo release may be determined by measuring the plasma levels of the active agent or metabolites thereof (i.e., AUC, Cₘₐₓ).

In one embodiment, the invention provides a sequestering subunit comprising an opioid antagonist and a blocking agent, wherein the blocking agent substantially prevents release of the opioid antagonist from the sequestering subunit in the gastrointestinal tract for a time period that is greater than 24 hours. This sequestering subunit is incorporated into a single pharmaceutical unit that also includes an opioid agonist. The pharmaceutical unit thus includes a core portion to which the opioid antagonist is applied. A seal coat is then optionally applied upon the antagonist. Upon the seal coat is then applied a composition comprising the pharmaceutically active agent in a releasable form. An additional layer containing the same or a different blocking agent may then be optionally applied such that the opioid agonist is released in the digestive tract over time (i.e., controlled release). Alternatively, the opioid agonist layer may be in an immediate release form. Thus, the opioid antagonist and the opioid agonist are both contained within a single pharmaceutical unit, which is typically in the form of a bead.

The term “sequestering subunit” as used herein refers to any pharmaceutical unit (e.g., bead or pellet) comprising a means for containing an antagonist and preventing or substantially preventing the release thereof in the gastrointestinal tract when intact, i.e., when not tampered with. The term “blocking agent” as used herein refers to the means by which the sequestering subunit is able to prevent substantially the antagonist from being released. The blocking agent may be a sequestering polymer, for instance, as described in greater detail below.

The terms “substantially prevents,” “prevents,” or any words stemming therefrom, as used herein, means that the antagonist is substantially not released from the sequestering subunit in the gastrointestinal tract. By “substantially not released” is meant that the antagonist may be released in a small amount, but the amount released does not affect or does not significantly affect the analgesic efficacy when the dosage form is orally administered to a host, e.g., a mammal (e.g., a human), as intended. The terms “substantially prevents,” “prevents,” or any words stemming therefrom, as used herein, does not necessarily imply a complete or 100% prevention. Rather, there are varying degrees of prevention of which one of ordinary skill in the art recognizes as having a potential benefit. In this regard, the blocking agent substantially prevents or prevents the release of the antagonist to the extent that at least about 80% of the antagonist is prevented from being released from the sequestering subunit in the gastrointestinal tract for a time period that is greater than 24 hours. Preferably, the blocking agent prevents release of at least about 90% of the antagonist from the sequestering subunit in the gastrointestinal tract for a time period that is greater than 24 hours. More preferably, the blocking agent prevents release of at least about 95% of the antagonist from the sequestering subunit. Most preferably, the blocking agent prevents release of at least about 99% of the antagonist from the sequestering subunit in the gastrointestinal tract for a time period that is greater than 24 hours.

For purposes of this invention, the amount of the antagonist released after oral administration can be measured in-vitro by dissolution testing as described in the United States Pharmacopeia (USP26) in chapter <711> Dissolution. For example, using 900 mL of 0.1 N HCl, Apparatus 2 (Paddle), 75 rpm, at 37° C. to measure release at various times from the dosage unit. Other methods of measuring the release of an antagonist from a sequestering subunit over a given period of time are known in the art (see, e.g., USP26).

Without being bound to any particular theory, it is believed that the sequestering subunit of the invention overcomes the limitations of the sequestered forms of an antagonist known in the art in that the sequestering subunit of the invention reduces osmotically-driven release of the antagonist from the sequestering subunit. Furthermore, it is believed that the present inventive sequestering subunit reduces the release of the antagonist for a longer period of time (e.g., greater than 24 hours) in comparison to the sequestered forms of antagonists known in the art. The fact that the sequestered subunit of the invention provides a longer prevention of release of the antagonist is particularly relevant, since precipitated withdrawal could occur after the time for which the therapeutic agent is released and acts. It is well known that the gastrointestinal tract transit time for individuals varies greatly within the population. Hence, the residue of the dosage form may be retained in the tract for longer than 24 hours, and in some cases for longer than 48 hours. It is further well known that opioid analgesics cause decreased bowel motility, further prolonging gastrointestinal tract transit time. Currently, sustained-release forms having an effect over a 24 hour time period have been approved by the Food and Drug Administration. In this regard, the present inventive sequestering subunit provides prevention of release of the antagonist for a time period that is greater than 24 hours when the sequestering subunit has not been tampered.

The sequestering subunit of the invention is designed to prevent substantially the release of the antagonist when intact. By “intact” is meant that a dosage form has not undergone tampering. As such, the antagonist and agonist are separated from one another within the intact dosage form. The term “tampering” is meant to include any manipulation by mechanical, thermal and/or chemical means, which changes the physical properties of the dosage form. The tampering can be, for example, crushing (e.g., by mortal and pestle), shearing, grinding, chewing, dissolution in a solvent, heating (for example, greater than about 45° C.), or any combination thereof. When the sequestering subunit of the invention has been tampered with, the antagonist is immediately released from the sequestering subunit. A dosage form that has been tampered with such that the antagonist has been released therefrom is considered “substantially disrupted” where, upon administration of the dosage form to a subject (e.g., a human being), the antagonist inhibits or otherwise interferes with the activity of the agonist in the subject including interfering with the agonist's ability to induce respiratory depression. Whether or not the antagonist is inhibiting or otherwise interfering with the activity of the agonist may be determined using any of a pharmacodynamic (PD) or pharmacokinetic (PK) measurements available to one of skill in the art, including but not limited to those described herein. If the antagonist is interfering with the action of the agonist, a statistically significant difference in the measurements of one or more PD or PK measurements is typically observed between dosage forms.

By “subunit” is meant to include a composition, mixture, particle; etc., that can provide a dosage form (e.g., an oral dosage form) when combined with another subunit. The subunit can be in the form of a bead, pellet, granule, spheroid, or the like, and can be combined with additional same or different subunits, in the form of a capsule, tablet or the like, to provide a dosage form, e.g., an oral dosage form. The subunit may also be part of a larger, single unit, forming part of that unit, such as a layer. For instance, the subunit may be a core coated with an antagonist and a scal coat; this subunit may then be coated with additional compositions including a pharmaceutically active agent such as an opioid agonist.

By “antagonist of a therapeutic agent” is meant any drug or molecule, naturally-occurring or synthetic that binds to the same target molecule (e.g., a receptor) of the therapeutic agent, yet does not produce a therapeutic, intracellular, or in vivo response. In this regard, the antagonist of a therapeutic agent binds to the receptor of the therapeutic agent, thereby preventing the therapeutic agent from acting on the receptor. In the case of opioids, an antagonist may prevent respiratory depression.

Standard pharmacodynamic (PD) and pharmacokinetic (PK) measurements may be used to compare the effects of different dosage forms (e.g., intact vs. “tampered with” or “substantially disrupted”) on a subject or to determine if a dosage form has been tampered with or rendered substantially disrupted. Standard measurements include, for example, known PD standards or scales including but not limited to one or more of VAS-Drug Liking (Balster & Bigelow, 2003; Griffiths et al. 2003), VAS-Overall Drug Liking, ARCI short form (Martin et al., 1971), Colc/ARCI (Cole et al., 1982), Colc/ARCI-Stimulation Euphoria, Subjective Drug Value (Girffiths, et al, 1993; Griffiths, et al. 1996), Cole/ARCI Abuse Potential, ARCI-Morphine Benzedrine Group (MBG), VAS-Good Effects, VAS-Feeling High, VAS-Bad Effects, VAS-Feel Sick, VAS-Nausea, ARCI-LSD, Cole/ARCI-Unpleasantness-Physical, Cole/ARCI-Unpleasantness-Dysphoria, VAS-Any Effects, VAS-Dizzincss, ARCI-Amphetamine, ARCI-BG, Cole/ARCI-Stimulation-Motor, VAS-Sleepy, ARCI-PCAG, Cole/ARCI-Sedation-Mental, Sedation-Motor, and/or pupillometry (Knaggs, et al. 2004), among others. Measurements may include mean and/or median Area Under the Effect Curve 0-2 h Post-dose (AUE₍₀₋₂ₕ₎), Area Under the Effect Curve 0-8 h Post-dose (AUE₍₀₋₈ₕ₎), Area Under the Effect Curve 0-24 h Post-dose (AUE₍₀₋₂₄ₕ₎), Apparent Post-dose Pupil Diameter (e.g., PCₘᵢₙ, PAOC₍₀₋₂ₕ₎, PAOC₍₀₋₈ₕ₎, PAOC₍₀₋₂₄ₕ₎), Raw Score at 1.5 hours Post-dose (HR1.5), maximum effect (Eₘₐₓ), Time to Reach the Maximum Effect (TEₘₐₓ). Particularly informative are Emax measurements for VAS-Drug Liking, VAS-Overall Drug Liking, Cole/ARCI-Stimulation Euphoria, Subjective Drug Value, Cole/ARCI Abuse Potential, ARCI-MBG, VAS-Good Effects, VAS-Feeling High, and pupillometry.

For the compositions described herein, PK measurements relating to the release of morphine and naltrexone may be useful. Measurements of morphine, naltrexone and/or 6-β-naltrexol levels in the blood (e.g., plasma) or patients to whom various dosage forms have been administered are useful. Specific PK parameters that may be measured include, for example, mean and/or median peak concentration in Maximum Plasma Concentration (Cₘₐₓ), time to peak concentration (Tₘₐₓ), elimination rate constant (λ_{z}), terminal half-life (T_{1/2}), area under the concentration-time curve 0 hours post-dose to 8 hours post-dose (AUC₀₋₈ₕ) (pg*h/ml), area under the concentration-time curve from time-zero to the time of the last quantifiable concentration (AUCₗₐₛₜ) (pg*h/ml), and area under the plasma concentration time curve from time-zero extrapolated to infinity (AUC_{inf}) (pg*h/ml), elimination rate (ke) (1/h), clearance (L/h), and/or volume of distribution (L). Samples (e.g., blood) may be withdrawn from those to whom the dosage form has been administered at various time points (e.g., approximately any of 0.5, 1, 1.5, 2, 3, 4, 6, 8, 10, 12 hours after administration). Where the sample is blood, plasma may be prepared from such samples using standard techniques and the measurements may be made therefrom. Mean and/or median plasma measurements may then be calculated and compared for the various dosage forms.

In certain embodiments, one or more of such standard measurements observed following administration of a dosage form may be considered different, reduced or increased from that observed following administration of a different dosage form where the difference between the effects of the dosage forms differs by about any of the following ranges: 5-10%, 10-15%, 15-20%, 10-20%, 20-25%, 25-30%, 20-30%, 30-35%, 35-40%, 30-40%, 40-45%, 45-50%, 40-50%, 50-55%, 55-60%, 50-60%, 60-65%, 65-70%, 60-70%, 70-75%, 75-80%, 70-80%, 80-85%, 85-90%, 80-90%, 90-95%, 95-100%, and 90-100%. In some embodiments, measurements may be considered “similar” to one another where there is less than about any of 0%, 5%, 10%, 15%, 20% or 25% difference. The difference may also be expressed as a fraction or ratio. For instance, the measurement observed for an intact dosage or substantially disrupted dosage form may be expressed as, for instance, approximately any of ½ (one-half), ⅓ (one-third), ¼ (one-fourth), ⅕ (one-fifth), ⅙ (one sixth), 1/7 (one-seventh), ⅛ (one-eighth), 1/9 (one-ninth), 1/10 (one-tenth), 1/20 (one-twentieth), 1/30 (one-thirtieth), 1/40 (one-fourtieth), 1/50 (one-fiftieth), 1/100 (one-one hundredth), 1/250 (one-two hundred fiftieth), 1/500 (one-five hundredth), or 1/1000 one-one thousandth) of that of the substantially disrupted or intact dosage form, respectively. The difference may also be expressed as a ratio (e.g., approximately any of 0.001:1, 0.005:1, 0.01:1, 0.1, 0.2:1, 0.3:1, 0.4:1, 0.5:1, 0.6:1, 0.7:1, 0.8:1, 0.9:1, 1:1, 1:2, 1:3, 1:4, 1:5, 1:6, 1:7, 1:8, 1:9, or 1:10).

To be regarded as “significant”, “statistically different”, “significantly reduced” or “significantly higher”, for example, the numerical values or measurements relating to the observed difference(s) may be subjected to statistical analysis. Baseline measures may be collected and significant baseline effect may be found. The treatment effect may be evaluated after the baseline covariate adjustment was made in the analysis of covariance (ANCOVA) model. The model may include treatment, period, and sequence as the fixed effects and subjects are nested within sequence as a random effect. For pharmacodynamic measures that have pre-dose values, the model may include the pre-dose baseline value as a covariate. The linear mixed effect model may be based on the per protocol population. A 5% Type I error rate with a p-value less than 0.05 may be considered “statistically significant” for all individual hypothesis tests. All statistical tests may be performed using two-tailed significance criteria. For each of the main effects, the null hypothesis may be “there was no main effect,” and the alternative hypothesis may be “there was a main effect.” For each of the contrasts, the null hypothesis may be “there was no effect difference between the tested pair,” and the alternative hypothesis may be “there was effect difference between the tested pair.” The Benjamin and Hochberg procedure may be used to control for Type I error arising from multiple treatment comparisons for all primary endpoints.

Statistical significance may also be measured using Analysis of variance (ANOVA) and the Schuimann's two one-sided t-test procedures at the 5% significance level. For instance, the log-transformed PK exposure parameters Cmax, AUCₗₐₛₜ and AUC_{inf} may be compared to determine statistically significant differences between dosage forms. The 90% confidence interval for the ratio of the geometric means (Test/Reference) may be calculated. In certain embodiments, dosage forms may be said to be “bioequivalent” or “bioequivalence” may be declared if the lower and upper confidence intervals of the log-transformed parameters are within about any of 70-125%, 80%-125%, or 90-125% of one another. A bioequivalent or bioequivalence is preferably declared where the lower and upper confidence intervals of the log-transformed parameters are about 80%-125%.

The release of morphine, naltrexone and 6-β-naltrexol from the different compositions in vitro may be determined using standard dissolution testing techniques such as those described in the United States Pharmacopeia (USP26) in chapter <711> Dissolution (e.g., 900 mL of 0.1 N HCl, Apparatus 2 (Paddle), 75 rpm, at 37° C.; 37° C. and 100 rpm) or 72 hours in a suitable buffer such as 500 mL of 0.05M pH 7.5 phosphate buffer) to measure release at various times from the dosage unit. Other methods of measuring the release of an antagonist from a sequestering subunit over a given period of time are known in the art (see, e.g., USP26) and may also be utilized. Such assays may also be used in modified form by, for example, using a buffer system containing a surfactant (e.g., 72 hrs in 0.2% Triton X-100/0.2% sodium acetate/0.002N HCl, pH 5.5). Blood levels (including, for example, plasma levels) of morphine, naltrexone and 6-β-naltrexol may be measured using standard techniques.

The antagonist can be any agent that negates the effect of the therapeutic agent or produces a diminution of deleterious effects of opioid induced respiratory depression.

The therapeutic agent can be an opioid agonist. By “opioid” is meant to include a drug, hormone, or other chemical or biological substance, natural or synthetic, having a sedative, narcotic, or otherwise similar effect(s) to those containing opium or its natural or synthetic derivatives. By “opioid agonist,” sometimes used herein interchangeably with terms “opioid” and “opioid analgesic,” is meant to include one or more opioid agonists, either alone or in combination, and is further meant to include the base of the opioid, mixed or combined agonist-antagonists, partial agonists, pharmaceutically acceptable salts thereof, stereoisomers thereof, ethers thereof, esters thereof, and combinations thereof.

Opioid agonists include, for example, alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydroetorphine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenazocine, phenomorphan, phenoperidine, piminodine, piritramide, propheptazine, promedol, properidine, propiram, propoxyphene, sufentanil, tramadol, tilidine, derivatives or complexes thereof, pharmaceutically acceptable salts thereof, and combinations thereof. Preferably, the opioid agonist is selected from the group consisting of hydrocodone, hydromorphone, oxycodone, dihydrocodeine, codeine, dihydromorphine, morphine, buprenorphine, derivatives or complexes thereof, pharmaceutically acceptable salts thereof, and combinations thereof. Most preferably, the opioid agonist is morphine, hydromorphone, oxycodone or hydrocodone. In a preferred embodiment, the opioid agonist comprises oxycodone or hydrocodone and is present in the dosage form in an amount of about 15 to about 45 mg, and the opioid antagonist comprises naltrexone and is present in the dosage form in an amount of about 0.5 to about 5 mg. Equianalgesic calculated doses (mg) of these opioids, in comparison to a 15 mg dose of hydrocodone, are as follows: oxycodone (13.5 mg); codeine (90.0 mg), hydrocodone (15.0 mg), hydromorphone (3.375 mg), levorphanol (1.8 mg), meperidine (15.0 mg), methadone (9.0 mg), and morphine (27.0).

Hydrocodone is a semisynthetic narcotic analgesic and antitussive with multiple nervous system and gastrointestinal actions. Chemically, hydrocodone is 4,5-epoxy-3-methoxy-17-methylmorphinan-6-one, and is also known as dihydrocodeinone. Like other opioids, hydrocodone can be habit-forming and can produce drug dependence of the morphine type. Like other opium derivatives, excess doses of hydrocodone will depress respiration.

Oral hydrocodone is also available in Europe (e.g., Belgium, Germany, Greece, Italy, Luxembourg, Norway and Switzerland) as an antitussive agent. A parenteral formulation is also available in Germany as an antitussive agent. For use as an analgesic, hydrocodone bitartrate is commonly available in the United States only as a fixed combination with non-opiate drugs (e.g., ibuprofen, acetaminophen, aspirin; etc.) for relief of moderate to moderately severe pain.

In embodiments in which the opioid agonist comprises hydrocodone, the sustained-release oral dosage forms can include analgesic doses from about 8 mg to about 50 mg of hydrocodone per dosage unit. In sustained-release oral dosage forms where hydromorphone is the therapeutically active opioid, it is included in an amount from about 2 mg to about 64 mg hydromorphone hydrochloride. In another embodiment, the opioid agonist comprises morphine, and the sustained-release oral dosage forms of the invention include from about 2.5 mg to about 800 mg morphine, by weight. In yet another embodiment, the opioid agonist comprises oxycodone and the sustained-release oral dosage forms include from about 2.5 mg to about 800 mg oxycodone.

In a preferred embodiment, the opioid antagonist comprises naltrexone or a salt of naltrexone. In the treatment of patients previously addicted to opioids, naltrexone has been used in large oral doses (over 100 mg) to prevent cuphorigenic effects of opioid agonists. Naltrexone has been reported to exert strong preferential blocking action against mu over delta sites. Naltrexone is known as a synthetic congener of oxymorphone with no opioid agonist properties, and differs in structure from oxymorphone by the replacement of the methyl group located on the nitrogen atom of oxymorphone with a cyclopropylmethyl group. The hydrochloride salt of naltrexone is soluble in water up to about 100 mg/cc. The pharmacological and pharmacokinetic properties of naltrexone have been evaluated in multiple animal and clinical studies. See, e.g., Gonzalez et al. Drugs 35:192-213 (1988). Following oral administration, naltrexone is rapidly absorbed (within 1 hour) and has an oral bioavailability ranging from 5-40%. Naltrexone's protein binding is approximately 21% and the volume of distribution following single-dose administration is 16.1 L/kg.

Naltrexone is commercially available in tablet form (Revia®, DuPont (Wilmington, Del.)) for the treatment of alcohol dependence and for the blockade of exogenously administered opioids. Sec, e.g., Revia (naltrexone hydrochloride tablets), Physician's Desk Reference, 51^{st} ed., Montvale, N.J.; and Medical Economics 51:957-959 (1997). A dosage of 50 mg Revia® blocks the pharmacological effects of 25 mg IV administered heroin for up to 24 hours. It is known that, when coadministered with morphine, heroin or other opioids on a chronic basis, naltrexone blocks the development of physical dependence to opioids. It is believed that the method by which naltrexone blocks the effects of heroin is by competitively binding at the opioid receptors. Naltrexone has been used to treat narcotic addiction by complete blockade of the effects of opioids. It has been found that the most successful use of naltrexone for a narcotic addiction is with narcotic addicts having good prognosis, as part of a comprehensive occupational or rehabilitative program involving behavioral control or other compliance-enhancing methods. For treatment of narcotic dependence with naltrexone, it is desirable that the patient be opioid-free for at least 7-10 days. The initial dosage of naltrexone for such purposes has typically been about 25 mg, and if no withdrawal signs occur, the dosage may be increased to 50 mg per day. A daily dosage of 50 mg is considered to produce adequate clinical blockade of the actions of parenterally administered opioids. Naltrexone also has been used for the treatment of alcoholism as an adjunct with social and psychotherapeutic methods. Other preferred opioid antagonists include, for example, cyclazocine and naltrexone, both of which have cyclopropylmethyl substitutions on the nitrogen, retain much of their efficacy by the oral route, and last longer, with durations approaching 24 hours after oral administration.

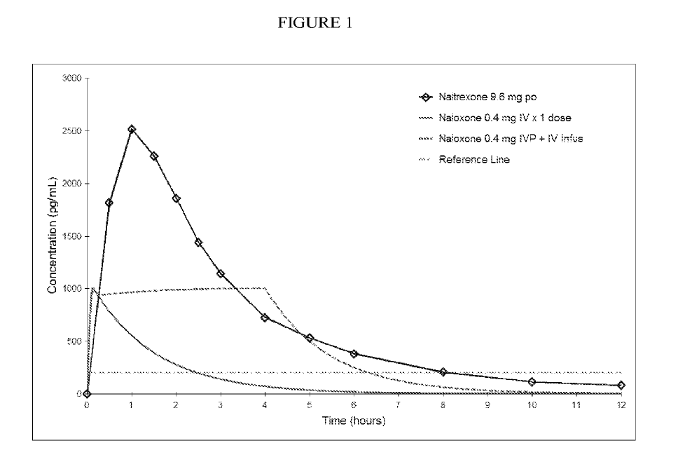

Based on estimates of naloxone systemic clearance and half-life, the naloxone concentration profiles following an IV injection of 0.4 mg with and without a continuous infusion of naloxone over 4 hours can be simulated as shown in FIG. 1, with the solid red line representing the plasma naloxone concentration profile following a single bolus dose and the dashed line representing the profile following the bolus dose plus and the continuous infusion over 4 hours.

Contrasted with the therapeutic concentration profiles for naloxone is the concentration profile naltrexone if all of the drug were released from an 80 mg dose of ALO-02 (oxycodone 80 mg) containing 12% naltrexone. Theoretically, with peak naltrexone concentrations reaching as high as 2500 pg/mL, the amount of naltrexone reaching the systemic circulation acts as a rescue medication if a oxycodone sequestered naltrexone formulation were chewed or crushed in an attempt to misuse the formulation. (Gonzalez J P and Brogden R N. Naltrexone: A review of its pharmacodynamic and pharmacokinetic properties and therapeutic efficacy in the management of opioid dependence. Drugs. 1988; 35:192-213; Verebey K, Volavka J, Mute S J, and Resnick RB. Naltrexone: Disposition, metabolism, and effects after acute and chronic dosing. Clin Pharm and Ther. 1976; 20(3):315-28; Willette R E and Barnett G. Narcotic antagonists: naltrexone pharmacochemistry and sustained-release preparation. Department of Health and Human Services. National Institute on Drug Abuse (NIDA), Division of Research. NIDA Research Monotraph 28, 1981.)

The opioid agonist/naltrexone ratio that will attenuate opioid induced respiratory depression will depend in part on the opioid agonist. Ideally, the ratio is such that if the formulation is tampered with the amount of naltrexone released upon tampering will prevent the induction of respiratory depression when the tampered formulation is administered to a human. The formulations of the present invention also include opioid agonist/naltrexone ratios which reduce the severity of the respiratory depression induced by opioid abuse. In certain embodiments the ratio of oxycodone to naltrexone in the composition is from about 2% to about 30%. In another embodiment the ratio of oxycodone to naltrexone in the composition is from about 2% to about 20%. In an embodiment the ratio of oxycodone to naltrexone in the composition is from about from 2:1 (50%) to about 50:1 (2%). In a preferred embodiment the ratio of oxycodone to naltrexone in the composition is from about 5:1 (20%) to about 25:1 (4%). In a preferred embodiment the ratio of oxycodone to naltrexone in the composition is from about 10:1 (10%) to about 20:3 (15%).

In an embodiment the ratio of hydrocodone to naltrexone in the composition is from about from 1:1 (100%) to about 100:1 (1%). In a preferred embodiment the ratio of hydrocodone to naltrexone in the composition is from about 5:1 (20%) to about 25:1 (4%). In a preferred embodiment the ratio of hydrocodone to naltrexone in the composition is from about 10:1 (10%) to about 20:3 (15%).

In an embodiment the ratio of morphine to naltrexone in the composition is from about from 1:1 (100%) to about 100:1 (1%). In a preferred embodiment the ratio of morphine to naltrexone in the composition is from about 5:1 (20%) to about 25:1 (4%). In a preferred embodiment the ratio of morphine to naltrexone in the composition is from about 50:1 (2%) to about 20:3 (15%).

Respiration is the exchange of oxygen and carbon dioxide. The adequacy of respiration can be measured in terms of maintenance of arterial carbon dioxide and oxygen tensions within the normal ranges. Ventilation is usually described in terms of alveolar ventilation sufficient to maintain the arterial CO₂ and O₂. Unfortunately continuous, non-invasive measurement of arterial blood gas tensions is unavailable. At best intermittent blood gas sampling is possible but this requires the placement of an invasive arterial line and may be considered clinically inappropriate in certain study populations. Therefore surrogates of arterial CO₂ and O₂ have been sought e.g. end-tidal CO₂ (the level of carbon dioxide in the air exhaled from the body, the normal values of which are 4% to 6%; that is equivalent to 35 to 45 mm Hg) and SpO₂ (Pulse oximetry provides estimates of arterial oxyhemoglobin saturation (SaO₂) by utilizing selected wavelengths of light to noninvasively determine the saturation of oxyhemoglobin), respectively.

Ventilation requires both an intact respiratory system (lung units, patent airway) and an intact neural drive (brainstem respiratory center, spinal cord). Physical components of ventilation can be measured (e.g. respiratory rate, tidal volume) and be reported either alone or in combination (minute ventilation=respiratory rate×tidal volume). Neural drive can be measured by measuring ventilatory response to induced hypoxia and/or hypercarbia. The respiratory rate can be difficult to measure by an observer, particularly at low or irregular rates. Indirect measurement of respiratory rate using changes in electrical impedance of the ECG can yield the respiratory rate, but these are prone to error. The measurement of end-tidal CO₂ trace is dependent upon a patent airway, as is tidal volume measurement by pneumotachograph.

The characteristic pattern of opioid-induced respiratory depression is a reduced respiratory rate (bradypnea) with deep, sighing ventilations. Patients will often be conscious but lack the drive to breathe. Once given verbal commands to breathe, the patient will comply and take breaths when instructed to do so. The loss of central respiratory drive is typical of opioids, but this feature is difficult to quantify.

The mean arterial carbon dioxide tension is 38 mmHg and does not vary with age. In contrast, the arterial oxygen tension does vary with age (typically 94 mmHg in the age range 20-29; 81 mmHg in the age range 60-69). Furthermore, arterial oxygen tension is significantly altered in the presence of supplemental oxygen. Therefore, it is important to state the inspired oxygen fraction whenever arterial oxygen tensions are reported. For respiratory research purposes, it is preferable to conduct the study with subjects breathing room air rather than supplemental oxygen.

If respiration is the maintenance of adequate arterial CO₂ and O₂ tensions, then respiratory depression can be defined as the failure to maintain those arterial CO₂ and O₂ tensions. Several papers have highlighted the difficulty in defining specific thresholds of respiratory depression as there is usually no access to arterial blood gas data and so other respiratory parameters are selected. There is currently no consensus as to which individual parameters or combination of parameters adequately constitute respiratory depression.

Therefore, for purposes of this application a primary threshold of respiratory depression may the development of hypercarbia, the physical condition of having the presence of an abnormally high level of carbon dioxide in the circulating blood (PaCO₂>45 mmHg). During clinically significant respiratory depression, hypercarbia usually occurs in combination with a reduction in ventilatory performance, often manifesting as any combination of a reduction in respiratory rate, reduction in end-tidal volume, reduction in minute volume, reduction in arterial pH, reduction in O₂ saturation and increase in end tital CO₂ (ET CO₂) or transcutaneious CO₂ levels. Attenuation of opioid induced respiratory depression with naltrexone may be evidenced by a significant reduction in P_{ET}CO₂, an increase in ventilator performance, an increase in pH, an increase in O₂ and an increase in the slope of the ventilation-P_{ET}CO₂ relationship based on the hypercapnic ventilatory response (HCVR). Attenuation of opioid induced respiratory depression can be defined as at least a 5% reduction in P_{ET}CO₂ or at least a 5% increase in ventilation or at least a 5% increase in the slope of the ventilation-P_{ET}CO₂ relationship based on the hypercapnic ventilatory response. In preferred embodiments attenuation of opioid induced respiratory depression will provide at least a 10% reduction in P_{ET}CO₂ or at least a 10% increase in ventilation or at least a 10% increase in the slope of the ventilation-P_{ET}CO₂ relationship based on the hypercapnic ventilatory response. In more preferred embodiments attenuation of opioid induced respiratory depression will provide at least a 20% reduction in P_{ET}CO₂ or at least a 20% increase in ventilation or at least a 20% increase in the slope of the ventilation-P_{ET}CO₂ relationship based on the hypercapnic ventilatory response.

Thus the present invention relates to opiate analgesic drug formulations and methods of administering those formulations in which respiratory depression is attenuated in a human when the formulation has been tampered with prior to administration to the human.

Further embodiments and characterizations of the present invention are provided in the following non-limiting examples.

### EXAMPLES

### Example 1

### Effects of i.v. Naltrexone on Morphine-Induced Respiratory Depression in Healthy Volunteers

The respiratory depression study is a double-blind, randomized, 4-way crossover study in healthy volunteers, male or female subjects between the ages of 21 and 35 years, inclusive, and in generally good health as determined by the Investigator.

In Part A Dosing Period I, following a 15-day Screening period, a cohort of 4 subjects meeting the study inclusion/exclusion requirements is enrolled and randomized in a 3:1 ratio to receive either morphine sulfate injection 10 mg (N=3) or placebo (N=1).

During each treatment period, each subject is admitted to the clinic unit on the evening of Day −1. On Day 1 the subject receives study drug(s) and undergoes the pharmacodynamic, pharmacokinetic, and safety assessment procedures. The subject remains in the clinic unit until the morning of Day 2 at which time they are discharged from clinical unit at the discretion of the Investigator.

At the completion of Part A Dosing Period 1, the Investigator and the Sponsor reviews the unblinded safety and PD endpoint data and determines the appropriateness of escalating the morphine sulfate dose to 20 mg.

If deemed medically safe and appropriate, a second cohort of 4 subjects is randomized in a 3:1 ratio to receive either morphine sulfate injection 20 mg (N=3) or placebo (N=1). At the completion of Dosing Period 2, the Investigator and the Sponsor reviews the unblinded safety and PD endpoint data and determines the appropriateness of escalating the morphine sulfate dose to 30 mg.

If deemed medically safe and appropriate, a third cohort of 4 subjects is randomized in a 3:1 ratio to receive either morphine sulfate 30 mg (N=3) or placebo (N=1). At the completion of Dosing Period 3, the Investigator and the Sponsor reviews the unblinded safety and PD endpoint data and make a determination about the appropriate dose of morphine sulfate injection to take into Phase B.

During each of the Dosing Periods in Part A (IA-IIIA), subjects are be confined to the clinical unit for approximately 40 hours (2 nights and 3 days) and each dosing period are separated by a washout period of at least 7 days.

A minimum of 4 and maximum of 12 subjects participate in Part A.

### Part B: Treatment Phase

Part B is a randomized, double-blind, placebo-controlled, 4-way crossover study in 12 healthy volunteers. Following a Part B 15-day Screening period, subjects meeting the study inclusion/exclusion requirements are enrolled and randomized to one of 4 treatment sequence groups (1-4) as shown below. Each subject receives all 4 treatments (A, B, C, and D), with each treatment separated by at least a 1-week washout period. The morphine sulfate injection dose utilized in Part B is a dose determined to be medically safe and appropriate in Part A.

TABLE 1
Treatment Scheme

| | |
|---|---|
| | Treatment Periods (I-IV) |
| Sequence | and Treatments (A-D) |

| Group | I | II | III | IV |
|---|---|---|---|---|
| 1 (N = 3) | C | A | D | B |
| 2 (N = 3) | A | B | C | D |
| 3 (N = 3) | B | D | A | C |
| 4 (N = 3) | D | C | B | A |

Treatment A: Morphine sulfate* i.v. + Placebo (saline) i.v.
Treatment B: Morphine sulfate* i.v. + Naltrexone* 4% i.v.
Treatment C: Morphine sulfate* i.v. + Naloxone* 4% i.v.
Treatment D: Placebo(saline) i.v. + Naltrexone* 4% i.v
*The dose of morphine sulfate (10, 20, or 30 mg) will be determined from Part A of the study. The dose of naltrexone HCl and naloxone HCl (antagonist) in Part B will be 4% of the morphine sulfate dose used in Part B (e.g. 10 mg of morphine with 0.4 mg of antagonist, 20 mg of morphine with 0.8 mg of antagonist, and 30 mg of morphine with 1.2 mg of antagonist)

During each treatment period, each subject is admitted to the clinical unit on the evening of Day −1. On Day 1 the subject receives study drug(s) and undergoes the pharmacodynamic, pharmacokinetic, and safety assessment procedures. The subject remains in the clinical unit until the morning of Day 2 at which time they are discharged from clinical unit at the discretion of the Investigator. Subjects remain in the clinical unit until the morning of Day 2 at which time they are discharged from unit at the discretion of the Investigator.

During each of the 4 treatment periods (I-IV) in Part B subjects are confined to the clinical unit for approximately 40 hours (2 nights and 3 days), and each treatment is separated by a washout period of at least 7 days. A final safety assessment is performed at End of Study.

Commercial suppliers are used to obtain intravenous solutions of morphine sulfate, and naloxone HCl and naltrexone. The intravenous dosing solutions are drawn into syringes and diluted with normal saline (0.9% sodium chloride for injection) so that the final volume of dosing solution of each drug will be the same: morphine sulfate=10 mg in 10 mL of saline; naltrexone=0.4 mg in 10 mL saline; naloxone=0.4 mg in 10 mL saline, and placebo=10 mL of saline. All study drugs (i.e., morphine+placebo; morphine+naltrexone; morphine+naloxone; and placebo+naltrexone) are administered intravenously, concurrently utilizing a bi-fuse device connected to ultra mini-volume tubing delivered by a syringe infusion pump. This method of delivery allows for any two medications to be injected simultaneously with minimal mixing thus reducing the risk of intravenous compatibility concerns. Each medication is infused over a 2-minute period of time. The time and events schedule for conduct of this study is presented in 02.

TABLE 2
Overall Schedule of Time and Events

| | Part A¹ (Morphine | | | | | | | | | |
|---|---|---|---|---|---|---|---|---|---|---|
| | Dose Selection Phase) | | | | Part B¹ (Treatment Phase) | | | | | End |
| | Screening | | | | Screening | | | | | of |
| | Phase | Period | Period | Period | Phase | | Period | Period | Period | Study |
| Study Procedures | (Part A) | IA | IIA | IIIA | (Part B) | Period I | II | III | IV | Phase² |
| Visit | 1A | 2A | 3A | 4A | 1 | 2 | 3 | 4 | 5 | 5 |
| Informed Consent | X | | | | X | | | | | |
| Inclusion/Exclusion | X | | | | X | | | | | |
| Criteria | | | | | | | | | | |
| Physical Examination³ | X | | | | X | | | | | X |
| Clinical Laboratory | X | | | | X | | | | | X |
| Tests⁴ | | | | | | | | | | |
| Viral Serology⁵ | X | | | | X | | | | | |
| Vital Signs⁶ | X | X | X | X | X | X | X | X | X | X |
| 12-Lead ECG | X | | | | X | | | | | |
| Serum Pregnancy Test | X | | | | X | | | | | X |
| (females) | | | | | | | | | | |
| Urine Pregnancy Test | | X | X | X | | X | X | X | X | |
| (females) | | | | | | | | | | |
| Concomitant Drug | X | X | X | X | X | X | X | X | X | X |
| Review | | | | | | | | | | |
| Pre-treatment with | | X | X | X | | X | X | X | X | |
| ondansetron 0.4 mg | | | | | | | | | | |
| i.v. 1-hour before | | | | | | | | | | |
| dosing study drug | | | | | | | | | | |
| Urine Drug Test | X | X | X | X | X | X | X | X | X | |
| Urine Alcohol Test | X | X | X | X | X | X | X | X | X | |
| Randomization | | X | X | X | X | | | | | |
| Admission to DCRU | | X | X | X | | X | X | X | X | |
| Transcutaneous carbon | | continuous monitoring for 6 | | | | continuous monitoring | | | | |
| dioxide/SenTec | | hours | | | | for 6 hours | | | | |
| Pulse Oximetry | | continuous monitoring for 6 | | | | continuos monitoring | | | | |
| | | hours | | | | for 6 hours | | | | |
| Cardiac Telemetry⁶ | | continuous monitoring for 6 | | | | continuous monitoring for | | | | X |
| | | hours | | | | 6 hours | | | | |
| Respiration Rate⁶ | | continuous monitoring for 6 | | | | continuous monitoring for | | | | |
| | | hours | | | | 6 hours | | | | |
| BIS Monitoring⁷ | | continuous monitoring for 6 | | | | continuous monitoring for | | | | |
| | | hours | | | | 6 hours | | | | |
| Pneumotachography⁸ | | X | X | X | | X | X | X | X | |
| Resp. Inductance | | X | X | X | | X | X | X | X | |
| Plethysmography⁸ | | | | | | | | | | |
| Hypercapnic | | X | X | X | | X | X | X | X | |
| ventilatory | | | | | | | | | | |
| challenge/response | | | | | | | | | | |
| (HCVR)⁹ | | | | | | | | | | |
| Study Drug | | X | X | X | | X | X | X | X | |
| Administration | | | | | | | | | | |
| Arterial Blood Gases¹⁰ | | X | X | X | | X | X | X | X | |
| PK Plasma Sampling¹¹ | | X | X | X | | X | X | X | X | |
| Pupillometry¹² | | X | X | X | | X | X | X | X | |
| Adverse Event | | X | X | X | | X | X | X | X | X |
| Assessment | | | | | | | | | | |

| | | | | | | | |
|---|---|---|---|---|---|---|---|
| Discharge from the | X | X | X | X | X | X | X |
| DCRU | | | | | | | |

¹Treatment Periods will be separated by a 7-day washout period between doses
²Defined as approximately 24 hours post-dose Part B Dosing Period IV.
³Physical exam will include height, weight, and BMI.
⁴Clinical laboratory tests will be performed.
⁵HIV-1, HIV-2, hepatitis B, and hepatitis C screening
⁶Vital signs (blood pressure, heart rate, respiratory rate) will be measured. During the Part A and B dosing periods, vital signs will be monitored continuously for the first 6-hours post dose. Oral temperature will be taken during Screening and at check-in prior to each dosing period (Parts A and B)..
⁷BIS monitoring will be done continuously until 6 hours post Part B dosing periods.
⁸Pneumotachography and respiratory inductance plethysmography (RIP) will be done.
⁹A hypercapnic ventilatory challenge will be performed at baseline (within 1 hour pre-dose) and at 1 and 4 hours post-dose. A HCVR will be assessed at baseline (within 1 hour pre-dose), at nadir of respiratory depression and following recovery of respiratory depression.
¹⁰Arterial blood gases will be determined.
¹¹PK sampling will be done.
¹²Pupillometry will be done.

As outlined in the Time and Events Schedule (Table 2), for Dosing Periods TA through IIIA (Part A) and I through IV (Part B), subjects will follow the procedures outlined below during each 40-hour stay in the Duke Clinical Research Unit (DCRU). Each treatment will be separated by at least a 1-week washout period between doses of study drug(s).

### Study Day −1 (Evening Prior to Dosing)

Subjects meeting entry criteria based on the screening evaluation will report to the DCRU at least 10 hours prior to dosing. Subjects may be offered a meal and/or a snack as appropriate depending on time of check-in. The procedures noted below will be performed:
Subjects will be assigned a Treatment Sequence according to the randomization schedule (Part B only).
Urine pregnancy test (females only).
Urine drug screen. The test must be negative for the subject to continue.
Urine alcohol test. The test must be negative for the subject to continue.
Determine concomitant drug use and record on the eCRF.
Vital signs including oral temperature.

All subjects will undergo a supervised fast for a minimum of 6 hours before treatment. Water will be allowed as desired except for 2 hours before and after dosing. During the inpatient periods, subjects will be supervised at all times. A staff physician will either be present or on call throughout the study.

### Treatment Day

Following a supervised overnight fast of at least 6 hours, the study procedures will begin. The subject will be confined to a bed at an approximately 35° angle for at least 6 hours during which time the subject will lie quietly and cooperate fully with the Investigator and staff responsible for administering the study drug(s), monitoring safety, and acquiring experimental data. Ondansetron 0.4 mg i.v. will be provided one hour prior to study drug dosing in Parts A and B. All study drug(s) will be administered intravenously and concurrently over a 2-minute period using a bi-fuse mini-pump device which is capable of infusing two drugs simultaneously.

The peumotachograph will be removed for 15 minutes every two hours during the six hour post dose (Parts A and B) period at which time the subject may be provided a full liquid diet as tolerated.

After the 6-hour time point, at the discretion of the Investigator, the study participant may ambulate as permitted by DCRU staff. At that time, the subject will be served a standard lunch. Thereafter, there will be no restrictions on water or walking and a standard dinner will be served during the evening. The subject will remain in the DCRU until 24 hours post-dose (Day 2) when the subject will be discharged after meeting the requirements of the study.

Each treatment will be separated by at least a 1-week washout period between doses.

### Pharmacodynamic Measurements

The following procedures are performed for each treatment described in Part A and Part B. All sampling times will be determined in relation to the time of the onset of infusion of the study drug(s).
Pneumotachographic measurements are done to determine minute ventilation, respiratory rate, end tidal volume and CO₂ at time: −30 minutes, −10, and −5 minutes prior to dosing (pre-dose baseline values) and at 5, 15, 30, and 45 minutes and 1, 1.5, 2, 2.5, 3, 3.5, 4, and 6 hours post-dose of study drug(s).
Intermittent sampling of arterial blood are done at time: −15 min (pre-dose) and at 5, 15, and 30 minutes and 1, 1.5, 2, 2.5, 3, 3.5, 4, 5, and 6 hours post-dose to measure arterial carbon dioxide levels (PaCO₂), arterial pH, and oxygen saturation (SaO₂).
Pulse oximetry is done continuously from −30 minutes pre-dose until 6 hours post-dose to monitor oxygen saturation (SpO₂). Likewise, cardiac telemetry is used to monitor heart rate and blood pressure, a SenTec device is used to continuously monitor transcutaneous carbon dioxide (PtcCO₂), and a bispectral index (BIS) monitor is used to monitor level of consciousness over the same time period. Measurements are recorded at −15 min (pre-dose) and at 5, 15, and 30 minutes and 1, 1.5, 2, 2.5, 3, 3.5, 4, 5, and 6, 8, 12, and 24 hours post-dose.
A SenTec device is used to continuously monitor transcutaneous carbon dioxide (PtcCO₂), and a bispectral index (BIS) monitor will be used to monitor level of consciousness over the same time period.
Pupillometry measurements are performed at −20 minutes prior to dosing and at 10, 20, and 40 minutes and 1, 1.5, 2, 2.5, 3, 3.5, 4, 5, 6, 8, 12, and 24 hours post dose.
Respiratory inductive plethysmography (RIP) is used as a secondary measure to monitor respiratory rate and minute volume from time −30 minutes pre-dose until 6 hours post dose.
The hypercapnic ventilatory response (HCVR) challenge is performed at baseline (within 1-hour pre-dose) and at 1 hour and 4 hours post-dose at the discretion of the Investigator. The hypercapnic ventilatory response is assessed at baseline, at nadir of respiratory depression and following recovery of respiratory depression.
Cardiac telemetry will be used continuously to monitor heart rate, blood pressure, respiratory rate from −30 minutes until 6 hours post dose. Thereafter, for time points 8, 12, and 24 hours post-dose, vital signs will be taken with the subject in the seated position with feet flat on the floor. The subject should be sitting quietly for approximately 2 minutes prior to obtaining blood pressure and heart rate measurements
Serial sampling of venous blood is done as described below.

### Pharmacokinetic Measurements

### Blood Sample Collection and Storage

Part A: During Part A of the study a total of up to 195 mL of blood (13 samples per treatment×5 mL per sample×3 treatments) is drawn for the purpose of quantitating the concentrations of morphine, M3G, and M6G in plasma. Blood samples are collected in appropriately labeled K₂-EDTA Vacutainer® (collection) tubes at time 0 (pre-dose) and at 0.25, 0.5, 1, 1.5, 2, 2.5, 3, 4, 6, 8, 12, and 24 hours post-dose. Neither naloxone nor naltrexone are assayed during this part of the study.

Immediately upon sampling, each blood collection tubes is gently inverted several times to insure that the anticoagulant is thoroughly mixed with the blood and then chilled in a cryoblock (or ice bath). Within 45 minutes after collection, the blood samples are centrifuged at 4° C. for 10 minutes at 3,000 RPM. Using appropriate pipetting techniques, the plasma from each sample is transferred to 2 polypropylene screw top transfer tubes (one primary and one back-up) labeled with study and subject information (i.e., name of sponsor, study number, subject ID, date, nominal time, analyte). The plasma samples are stored in an upright position at −20±10° C. or colder until assayed.

Part B: During Part B of the study a total of up to 520 mL of blood (13 samples per treatment×10 mL per sample×4 treatments) are drawn for the purpose of quantitating the concentrations of morphine and either naloxone or naltrexone and relevant metabolites (M3G, M6G, 6-β-naltrexol) in plasma. Blood samples are collected in appropriately labeled K₂-EDTA Vacutainer® (collection) tubes at time 0 (pre-dose) and at 0.25, 0.5, 1, 1.5, 2, 2.5, 3, 4, 6, 8, 12, and 24 hours post-dose.

Immediately upon sampling, each blood collection tubes is gently inverted several times to insure that the anticoagulant is thoroughly mixed with the blood and then chilled in a cryoblock (or ice bath). Within 45 minutes after collection, the blood samples are centrifuged at 4° C. for 10 minutes at 3,000 RPM. Using appropriate pipetting techniques, the plasma from each sample is transferred to 2 polypropylene screw top transfer tubes (one for morphine and one for naloxone/naltrexone) labeled with study and subject information (i.e., name of sponsor, study number, subject ID, date, nominal time, analyte). The plasma samples are stored in an upright position at −20±10° C. or colder until assayed.

Principal pharmacodynamic (PD) parameters of interest will include either the maximum effect (e.g., Eₘₐₓ for PaCO₂ and ET CO₂) or minimum effect (e.g., Eₘᵢₙ for MV, RR, ET CO₂, slope, and arterial pH) occurring within 4 hours of dosing study drug. Additional supportive parameters for PaCO₂, MV, and will include the area under the effect curve over time from baseline (time 0) to 1 hour post dose (AUE₀₋₁ₕ), 2 hours post dose (AUE₀₋₂ₕ), 3 hours post dose (AUE₀₋₃ₕ), 4 hours post dose (AUE₀₋₄ₕ), and 6 hours post dose (AUE₀₋₆ₕ), and the time to maximum effect (Tₘₐₓ).

Primary Endpoints
Peak arterial carbon dioxide (PaCO₂)

Secondary Endpoints
Minute ventilation (MV)
Respiratory rate
End-tidal CO₂ (ET CO₂)
Slope of the MV versus PaCO₂ curve (hypercapnic ventilatory response)
Arterial pH
Arterial O₂ saturation
Transcutaneous carbon dioxide level (PtcCO₂)
Pupillary diameter
Bispectral Index (BIS)

### Pharmacokinetic Endpoints

The following pharmacokinetic parameters will be calculated, where applicable, for morphine, morphine-3-glucuronide (M3G), morphine-6-glucuronide (M6G), naltrexone, 6-β-naltrexol, and naloxone:
Peak concentration (Cₘₐₓ) and time of peak concentration (Tₘₐₓ)
Area under the plasma concentration time curve (AUC)
Distribution and elimination half-lives (t½_{α}, and t½_{β}) and mean residence time (MRT)
Systemic clearance (CL)

### Example 2

### Effects of i.v. Naltrexone on Morphine-Induced Respiratory Depression in Non-Dependent Opioid Preferring Male Subjects

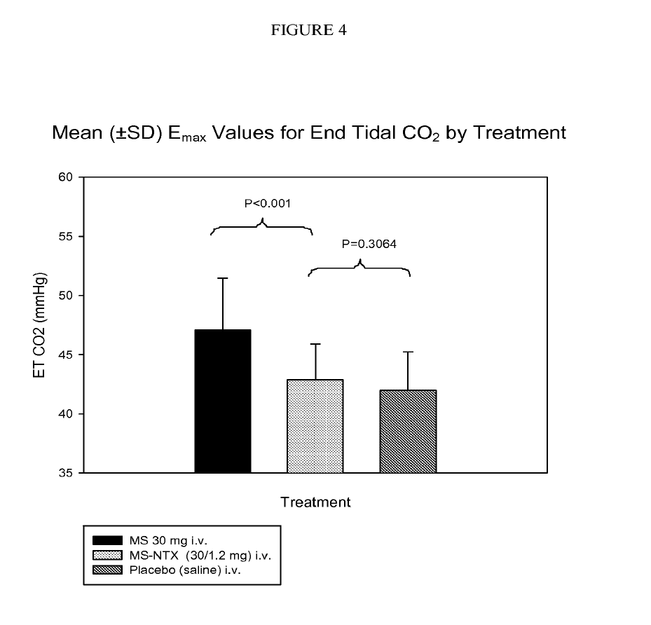

A single-dose, three-way crossover study in 28 opioid experienced, non-dependent male subjects indicate that naltrexone HCl 1.2 mg administered intravenously in combination with morphine sulfate 30 mg (Treatment A) significantly diminished morphine-induced respiratory depression compared with intravenous morphine sulfate 30 mg administered alone (Treatment B) or normal saline (placebo, Treatment C) (FIG. 4). All subjects were randomized to three sequential treatment doses using a cross-over design. Subjects received one dose on each dosing day in a double-blinded, cross-over manner (with a 6 day outpatient washout in between). An exploratory Analyses of EtCO₂ detected statistically significant differences in LS means across all treatment groups for Eₘₐₓ, and partial AUEs (p<0.0001). No difference was detected between the combination morphine+naltrexone and placebo groups in EtCO₂ levels (p=0.3064), which emphasizes the PD effect of morphine displacement on the μ-opioid receptor by naltrexone.

### Example 3

### Naltrexone Dose Ranging Study to Block Oxycodone-Induced Respiratory Depression

### Design and Investigational Plan:

The study is a randomized, double-blind, 5-way crossover study to evaluate the effects of oral naltrexone on oxycodone-induced respiratory depression in healthy male and female adult volunteers. The threshold dose of oxycodone that produces respiratory depression is investigated as a two part study. In Part A (Oxycodone Dose Response) escalating single doses of oxycodone immediate-release (IR) tablets will be administered orally to healthy volunteers to determine the appropriate dose of oxycodone that would safely produce distinguishable reductions in respiratory function (measured as reduced minute ventilation) in healthy volunteers. The oxycodone dose selected from Part A is used in Part B (Naltrexone Dose Response) in healthy volunteers to evaluate the naltrexone dose-response relationship with respect to attenuating oxycodone-induced respiratory depression.

### Screening

All subjects will be required to meet the study inclusion/exclusion criteria and complete the Screening requirements to participate in Part A or B of the study. Screening will be done no greater than 30 days prior to receiving study drug.

### Part A: Oxycodone Dose Response and Naltrexone “Test” Dose

Part A of the study is done in dose-escalating fashion in 6 healthy male or female adult volunteers. The study evaluates the safety and pharmacodynamic (PD) endpoints associated with a single 40 mg dose of JR oxycodone administered orally under unblended dosing conditions according to the study procedures described below. If the single 40 mg IR oxycodone dose is well tolerated, then a second treatment consisting of a single 80 mg dose of IR oxycodone is administered. However, if the 40 mg IR oxycodone dose is not well tolerated, the dose of oxycodone is reduced to 20 mg. All treatments will be separated by at least a 1-week washout period.

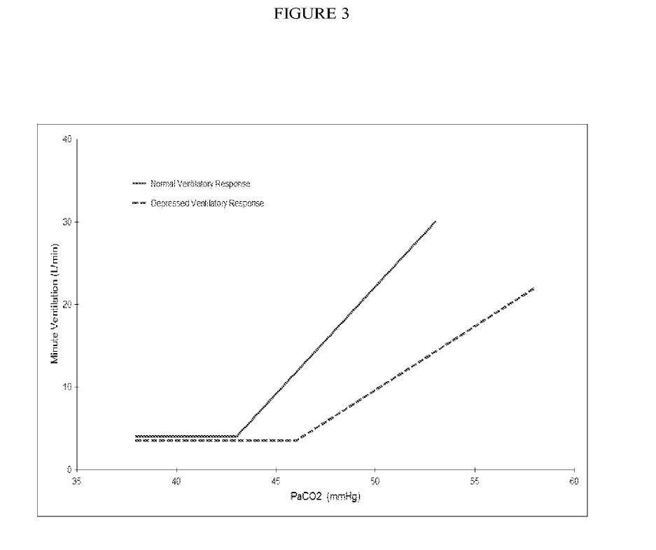

Safety and PD is evaluated prior to each dose escalation, however, the objective is to select the maximum oxycodone dose for Part B that could be safely tolerated and produce significant respiratory depression, defined as a depressed minute ventilation leading to a PaCO₂ value greater than 45 mmHg (FIG. 3). Once the appropriate oxycodone dose is identified, a 25 mg “test dose” of naltrexone is administered with the appropriate dose of oxycodone to determine administering naltrexone concomitantly with oxycodone attenuates oxycodone induced respiratory depression. Efficacy will be determined by an increase in minute ventilation, with an accompanying reduction in PaCO₂ and return to baseline values deemed “clinical reversal” of respiratory depression.

### Part B: Naltrexone Dose Response

Part B of the study is conducted in 12 healthy male and female adult volunteers, utilizing a randomized, five-way crossover design in which a standard dose of oxycodone (e.g., 80 mg) is co-administered with a variable (and blinded) dose of naltrexone, which is determined as a percent of the dose of oxycodone as described in Table 1 and below in “Study Drug(s) and Regimen”. Ultimately the dosage of naltrexone utilized for Treatments A-E depends on the dose of oxycodone (20 mg, 40 mg or 80 mg) selected from Part A of the study.

TABLE 1
Dose of Naltrexone by Treatment

| | Dose of | Dose of Naltrexone (mg) | | |
|---|---|---|---|---|
| Treatment | Naltrexone (%) | OXY 20 | OXY 40 | OXY 80 |
| A | 0 | 0 | 0 | 0 |
| B | 1.25% | 0.25 | 0.5 | 1.0 |
| C | 6.0% | 1.2 | 2.4 | 4.8 |
| D* | 12% | 2.4 | 4.8 | 9.6 |
| E | 20% | 6.25 | 12.5 | 25 |

*amount of naltrexone in ALO-02 (12% NTX)

### Study Procedures

During each dosing period subjects are admitted to the clinical research unit (CRU) on the evening of Day −1. On Day 1, following an overnight fast of at least 10 hours, the study procedures will begin. Baseline measurements of HCVR are performed under both hyperoxic and hypoxic challenge conditions. Likewise, baseline values of arterial carbon dioxide (PaCO₂), systemic pH, transcutaneous carbon dioxide (PtcCO₂), tidal volume and respiratory rate using respiratory inductive plethysmography (RIP) are established. Subjects are studied in the sitting position at a 35° angle for 6 hours, during which time they lie quietly and cooperate with the Investigator (and staff) responsible for controlling the study conditions, administering the study drugs, monitoring for safety, and acquiring data related to primary and secondary endpoints.

Study drug, consisting of a fixed dose of IR oxycodone± varying amounts of naltrexone in aqueous solution (Treatments A-E), is administered orally. Where applicable, certain PD assessments (PtcCO₂, respiratory rate, tidal volume) are followed and recorded continuously, while others (PaCO₂, systemic pH) are determined at specific time points (0, 0.25, 0.5, 1, 1.5, 2, 3, 4, 6, 8, 12, and 24 hours) according to the protocol. Likewise, serial sampling of venous blood is done at pre-dose (time 0), 0.25, 0.5, 1, 1.5, 2, 3, 4, 6, 8, 12, and 24 hours post dose for determination of oxycodone, naltrexone and related metabolite concentrations in plasma.

Transcutaneous carbon dioxide (PtcCO₂) is measured using an ear clip as a non-invasive means of estimating arterial PaCO₂. A cardiac monitor is used to measure basic vital signs. In addition, a VivoMetrics Life Shirt, containing elastic bands which measure the relative expansion of the thorax and abdomen during respiration, are worn by the subject to measure tidal volume and respiratory rate based on respiratory inductive plethysmography (RIP).

HCVR under hyperoxic and hypoxic challenge conditions is the most labor intensive procedure, taking up to 20 minutes to complete each test. It is done at time 0 (baseline), and at 1, 2, 4, and 6 hours post dose of study drug(s). The procedure involves securing a clear plastic RespirAct facemask to the subject's face and then controlling the delivery of a CO₂/O₂ gas mixture to the subject. This “rebreathing” technique is typically conducted under two different O₂ conditions, hypoxic (PO₂ 50 mmHg) and hyperoxic (PO₂ 150 mmHg). The hypoxic condition enhances peripheral chemoreceptor activity such that the ventilatory response remains the product of both central and peripheral chemoreceptor activity. In contrast, the hyperoxic condition suppresses peripheral chemoreceptor activity, thereby reflecting (or isolating) central chemoreceptor activity, which is the key component thought to be related to fatal opioid induced respiratory depression.

At 6 hours post dose the arterial line will be removed following satisfactory completion of the 6-hour HCVR test. At approximately 8 hours post dose subjects eat a standardized meal at the discretion of the Investigator. Thereafter, subjects can ambulate as desired. Subjects remain in the CRU until the morning of Day 2, at which time they are discharged from CRU at the discretion of the Investigator. Following a washout period of at least 7 days, subjects return to the CRU and repeat the study procedures described above during Treatment Periods II-V. A final safety assessment is done at End of Study. During each Treatment Period subjects are confined to the CRU for approximately 40 hours (2 nights and 3 days).

### Duration of Subject Participation:

Approximately 10 weeks including the Screening

### Study Population:

The study may enroll up to 24 subjects in an attempt to complete 6 subjects in Part A and 12 subjects in Part B.

### Study Drug(s) and Regimen:

Oxycodone is supplied as 5 mg immediate release tablets.

Naltrexone is supplied as 50 mg tablets which is used to prepare a “stock solution” of naltrexone (0.5 mg/mL) from which the doses of naltrexone are prepared. An example of the naltrexone treatments associated with an 80 mg dose of oxycodone are shown below.
Treatment A 0 mL of stock solution added to 150 mL of apple juice
Treatment B 2.0 mL of stock solution added to 148 mL of apple juice
Treatment C 9.6 mL of stock solution added to 140.4 mL of apple juice
Treatment D 19.2 mL of stock solution added to 130.8 mL of apple juice
Treatment E 50 mL of stock solution added to 100 mL of apple juice

Treatments A-E are followed with 90 mL of water for a total volume of 240 mL of fluid administered with each treatment.

### Statistical Methods:

### Sample Size

The study will enroll up to 24 subjects in an attempt to complete 6 subjects in Phase A and 12 subjects in Phase B.

### Analysis Populations

The safety population consists of all patients who took at least one dose of oxycodone. The PK/PD population consists of all patients who had undergone at least 6 hours of intensive PK sampling and PD evaluation.

Efficacy and/or PK/PD Analyses

The primary endpoints are minute ventilation, arterial PaCO₂, and slope of the ventilatory response to CO₂ curve. However, data for all PD and PK endpoints are summarized graphically and categorized by treatment using descriptive statistics, including mean, standard deviation, median, minimum, maximum, and 95% confidence interval (CI) for the evaluable population. Dose response of naltrexone is examined graphically. The time courses for all PD measures are presented graphically by treatment.

All PD endpoints are analyzed using a mixed-effect model for a crossover study, with treatment, period, and sequence as fixed effects and subject within sequence as a random effect. Statistical significance of all treatment differences are reported using two-tailed significance criteria.

### Safety Analyses

All AEs are coded to System Organ Class and Preferred Term using the Medical Dictionary for Regulatory Activities (MedDRA) and summarized by age group and treatment group. Treatment-emergent AEs are defined as AEs that commence on or after the time of oxycodone administration. Treatment emergent adverse events are summarized as follows:
Number of patients with AEs classified by System Organ Class and Preferred Term;
Number of patients with AEs by maximum intensity, System Organ Class and Preferred Term;
Number of patients with AEs by relationship to study drug, System Organ Class and Preferred Term;
Number of patients with SAEs classified by System Organ Class and Preferred Term.

Clinical laboratory test data (chemistry, hematology, and urinalysis) are summarized at the Screening Visit, the Post-Operative and Treatment Periods, where applicable, and the Post-Treatment Safety Follow-up Assessment. Vital signs are summarized at each time point.

### Example 4

### Effects of i.v. Naltrexone on Oxycondone-Induced Respiratory Depression in Healthy Volunteers

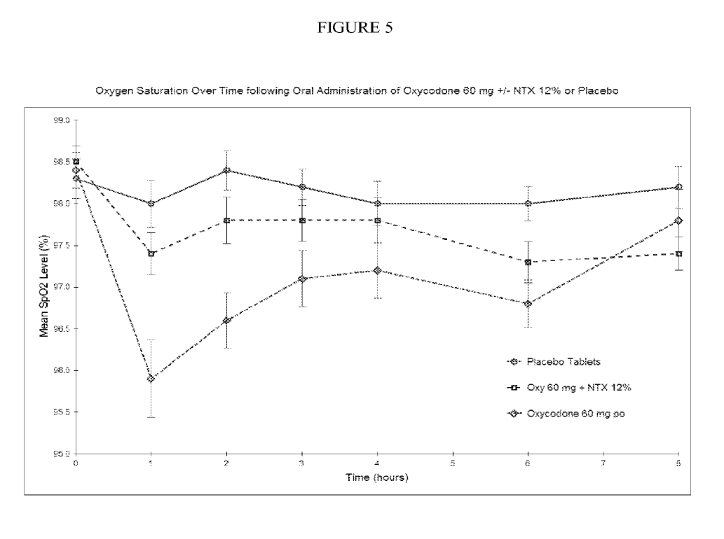

A randomized, placebo-controlled, six-way, crossover study to evaluate the effects naltrexone (12% w/w) on oxycodone-induced euphoria in opioid-experienced adult subjects was conducted. As a safety component of this study, pulse oximetry was monitored routinely to monitor for signs and symptoms of oxycodone-induced respiratory depression. FIG. 5 illustrates the mean (+/−SE) oxygen saturation (SpO₂) levels over time determined from pulse oximetry following oral administration of: oxycodone 60 mg; oxycodone 60 mg+naltrexone 7.2 mg (12%); and placebo.

The results indicate that, in addition to abating the euphoric effects of oxycodone 60 mg, naltrexone attenuated the respiratory depressant effects of oxycodone. The attenuation effect was most pronounced at the approximate peak time of oxycodone and naltrexone absorption, approximately 1-hour post dose.

What is claimed is:

1. An opiate analgesic drug formulation comprising a solid, controlled release, oral dosage form comprising a plurality of multi-layer pellets, each pellet comprising:
a) a water soluble core
b) an antagonist layer comprising naltrexone or a pharmaceutically acceptable salt of naltrexone coating the core;
c) a sequestering polymer layer coating the antagonist layer;
d) an agonist layer comprising an opioid or a pharmaceutically acceptable salt of the opioid coating the sequestering polymer layer, and
e) a controlled release layer coating the agonist layer
wherein substantially no naltrexone or a pharmaceutically acceptable salt of naltrexone is released when administered intact to a human and wherein respiratory depression which is induced in a human when the formulation has been tampered with prior to administration to the human is attenuated by the release of naltrexone or a pharmaceutically acceptable salt of naltrexone.

2. The formulation of claim 1 wherein the attenuation of respiratory depression is measured by reduction in P_{ET}CO₂.

3. The formulation of claim 2 wherein the reduction in P_{ET}CO₂ is at least 5%.

4. The formulation of claim 1 wherein attenuation of respiratory depression is measured by by an increase in oxygen saturation (SpO₂) levels.

5. The formulation of claim 1 wherein the opioid is morphine or a pharmaceutically acceptable salt of morphine.

6. The formulation of claim 1 wherein the opioid is oxycodone or a pharmaceutically acceptable salt of oxycodone.

7. Use of an opiate analgesic drug formulation in the manufacture of a medicament for attenuating drug-mediated respiratory depression in a human following administration of a respiratory depression-mediating opioid drug to the human, wherein the formulation comprises a plurality of multi-layer pellets, each pellet comprising:
a) a water soluble core
b) an antagonist layer comprising naltrexone or a pharmaceutically acceptable salt of naltrexone coating the core;
c) a sequestering polymer layer coating the antagonist layer;
d) an agonist layer comprising an opioid or a pharmaceutically acceptable salt of the opioid coating the sequestering polymer layer, and
e) a controlled release layer coating the agonist layer
wherein substantially no naltrexone or a pharmaceutically acceptable salt of naltrexone is released when administered intact to a human and wherein respiratory depression which is induced in a human when the formulation has been tampered with prior to administration to the human is attenuated by the release of naltrexone or a pharmaceutically acceptable salt of naltrexone.

8. The formulation of claim 7 wherein the attenuation of respiratory depression is measured by reduction in P_{ET}CO₂.

9. The formulation of claim 8 wherein the reduction in P_{ET}CO₂ is at least 5%.

10. The formulation of claim 7 wherein attenuation of respiratory depression is measured by an increase in oxygen saturation (SpO₂) levels.

11. The formulation of claim 7 wherein the opioid is morphine or a pharmaceutically acceptable salt of morphine.

12. The formulation of claim 7 wherein the opioid is oxycodone or a pharmaceutically acceptable salt of oxycodone.
